(19) United States
(12) Patent Application Publication
(10) Pub. No.: US 2025/0333679 A1
(43) Pub. Date: Oct. 30, 2025

(54) APPARATUS AND METHOD FOR CULTURING CELLS IN VITRO

(71) Applicant: Quantum NIL Corporation, Taipei City (TW)

(72) Inventors: Wang-Nang WANG, Taichung City (TW); Chung-Hsiang LIN, Taipei City (TW)

(21) Appl. No.: 18/889,635

(22) Filed: Sep. 19, 2024

Related U.S. Application Data
(60) Provisional application No. 63/638,550, filed on Apr. 25, 2024.

(51) Int. Cl. C12M 1/42

(52) U.S. Cl. CPC C12M 35/02

(57) ABSTRACT

An apparatus for culturing cells in vitro includes a culture carrier for culturing the cells and a pulse generating device which is capable of generating pulses simulating human heart pulses. The culture carrier includes a photonic quasicrystal pattern with multifold symmetry. The pulse generating device is disposed to transmit the pulses simulating human heart pulses to the cells on the culture carrier. A method for culturing cells in vitro includes the steps of: a) placing the cells in a culture carrier, the culture carrier including a photonic quasicrystal pattern with multifold symmetry; and b) transmitting pulses simulating human heart pulses to the cells on the culture carrier while culturing the cells on the culture carrier.

### CROSS-REFERENCE TO RELATED APPLICATION

This application claims the benefit of U.S. Provisional Patent Application No. 63/638,550, filed on Apr. 25, 2024, the entire disclosure of which is incorporated by reference herein.

### FIELD

The present disclosure relates to an apparatus for culturing cells in vitro. The present disclosure also relates to a method for culturing cells in vitro.

### BACKGROUND

In the field of cell cultivation and tissue engineering, various templates have been developed for the growth and differentiation of stem cells or organ cells. However, most of these templates are passive devices which lack the ability to mimic the dynamic physiological conditions required for optimal cell growth and/or differentiation.

### SUMMARY

Therefore, an object of the present disclosure is to provide an apparatus and a method for culturing cells in vitro that can alleviate at least one of the drawbacks of the prior art.

According to an aspect of the present disclosure, the apparatus for culturing cells in vitro includes a culture carrier for culturing the cells and a pulse generating device which is capable of generating pulses simulating human heart pulses. The culture carrier includes a photonic quasicrystal pattern with multifold symmetry. The pulse generating device is disposed to transmit the pulses simulating human heart pulses to the cells on the culture carrier.

According to another aspect of the present disclosure, the method for culturing cells in vitro includes the steps of:
a) placing the cells on a culture carrier, the culture carrier including a photonic quasicrystal pattern with multifold symmetry; and
b) transmitting pulses simulating human heart pulses to the cells on the culture carrier while culturing the cells on the culture carrier.

### BRIEF DESCRIPTION OF THE DRAWINGS

Other features and advantages of the present disclosure will become apparent in the following detailed description of the embodiment(s) with reference to the accompanying drawings. It is noted that various features may not be drawn to scale.

FIG. 1 is a schematic sectional view illustrating an apparatus for culturing cells in vitro including a culture carrier and a pulse generating device according to certain embodiments of the present disclosure.

FIG. 2 is a schematic perspective view illustrating first electrodes, second electrodes and third electrodes of the pulse generating device according to some embodiments of the present disclosure.

FIG. 3 is a schematic sectional view illustrating the culture carrier and the pulse generating device which are integrally formed according to some embodiments of the present disclosure.

FIG. 4 is a schematic view illustrating a photonic quasicrystal pattern of the culture carrier, which is a square-triangular tiling pattern with multifold symmetry.

FIG. 5 is a scanning electron microscope (SEM) image illustrating another photonic quasicrystal pattern of the culture carrier, which is a sunflower pattern.

FIG. 6 is an SEM image illustrating unit elements of the culture carrier which are in a form of rods with different dimensions.

FIGS. 7A and 7B are SEM images illustrating the unit elements which are in the forms of pillars and holes, respectively.

FIG. 8 is a schematic view illustrating the unit elements which are distributed as several photonic quasicrystal patterns with different arrangements of micrometer patterns and nanometer patterns.

### DETAILED DESCRIPTION

Before the present disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

In order to address the current limitations of passive devices for growth and/differentiation of stem cells and organ cells, the inventors of this application endeavored to developing a biomimetic approach in which pulses simulating heart pulses of humans are transmitted onto an active template which is formed with photonic quasicrystal patterns to imitate cellular arrangement in vivo, so as to permit the cells placed on the active template to be grown and/or differentiate under a dynamic physiological environment mimicking that of the human body (i.e., under the influence of heart-like electrical pulses and simulated cellular arrangement).

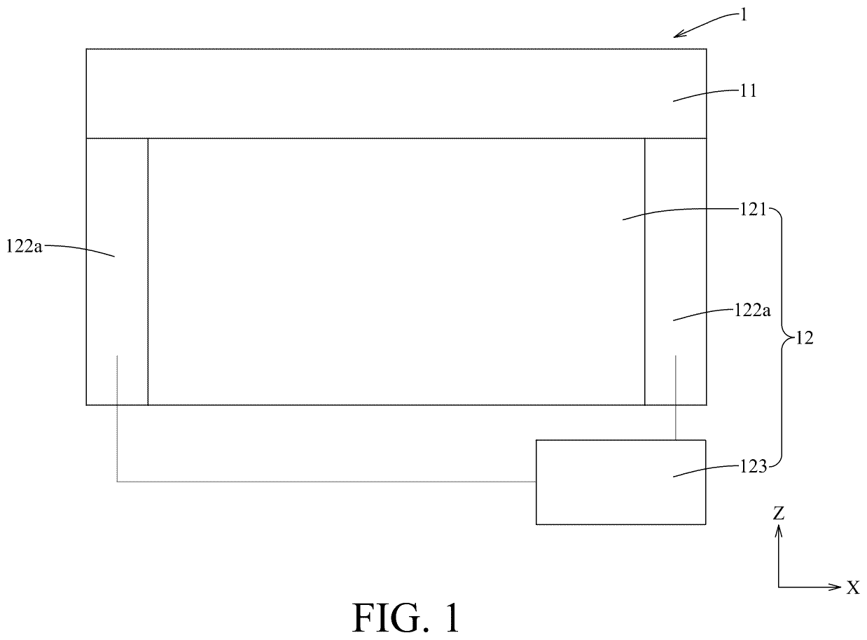

Referring to FIG. 1, an apparatus 1 for culturing cells (not shown) in vitro according to an embodiment of the present disclosure includes a culture carrier 11 for culturing the cells, and a pulse generating device 12. The culture carrier 11 includes a photonic quasicrystal pattern with multifold symmetry. The pulse generating device 12 is capable of generating pulses simulating human heart pulses and is disposed to transmit the pulses simulating human heart pulses to the cells on the culture carrier 11. Although not shown in figures, during culturing of the cells, a culture medium containing nutrients and growth factors is also added to the cells on the culture carrier 11.

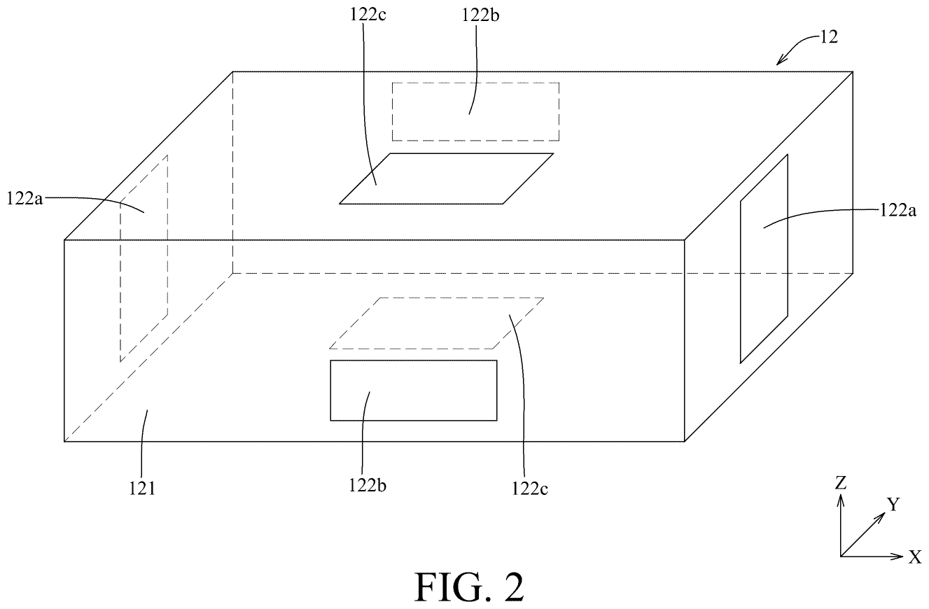

In some embodiments, as shown in FIGS. 1 and 2, the culture carrier 11 may be made of silicon and/or silicon coated with a material selected from biopolymers, proteins, polypeptides, other suitable materials, or combinations thereof, which have good affinity to the cells to be cultured on the culture carrier 11, so as to activate cell growth. Examples of the biopolymers include, but are not limited to, collagen, alginate, hyaluronic acid, and polyethylene glycol. In certain embodiments, the culture carrier 11 may be made of a piezoelectric material. Examples of the piezoelectric material may include, but are not limited to, lead zirconate titanate (PbZrTiO₃), zinc oxide (ZnO), gallium nitride (GaN), polyvinylidene fluoride (PVDF), barium titanate (BaTiO₃), sodium potassium niobate (KNaNbO₃), quartz, ceramic composites, berlinite (AlPO₄), lead titanate (PbTiO₃), lithium niobate (LiNbO₃), lithium tantalite (LiTaO₃), sodium tungstate (Na₂WO₃), bismuth ferrite (BiFeO₃), bismuth titanate (Bi₄Ti₃O₁₂), and boron nitride (BN). In some embodiments, the photonic quasicrystal pattern of the culture carrier 11 may be fabricated by nanoimprint technology, for example, nanoimprint lithography.

In some embodiments, piezoelectricity is utilized for generating the pulses simulating human heart pulses, while in other embodiments, other suitable means may be used for generating the pulses simulating human heart pulses. To generate the pulses simulating human heart pulses using the piezoelectricity, the pulse generating device 12 may include a piezoelectric element 121, two first electrodes 122a connected to the piezoelectric element 121 and disposed opposite to each other in a first direction (X), and an actuator 123 connected to the piezoelectric element 121 through the two first electrodes 122a. The actuator 123 is capable of generating an adjustable pulse-width modulation (PWM) signal so as to permit the piezoelectric element 121 to generate the pulses simulating human heart pulses. Since the human heart rate is not constant (i.e., heart rate variability), the PWM signal may be varied with time so as to provide a realistic representation of human heart pulses.

It is noted that the various cells and proteins may also process piezoelectric property, and hence, the pulses simulating human heart pulses may also activate growth and/or differentiation of the cells in the culture medium.

In certain embodiments, the piezoelectric element 121 is made of the piezoelectric material as mentioned in the foregoing, and is disposed beneath the culture carrier 11.

When electrical energy is applied to a piezoelectric material, the piezoelectric material will convert the electrical energy into mechanical energy. Referring again to FIG. 1, by utilizing such property of the piezoelectric material, when the electrical energy in the form of the PWM signal is applied to the piezoelectric element 121 through the two first electrodes 122a, such electrical energy will result in repetitive contraction and expansion of the piezoelectric element 121 along the first direction (X), thereby generating the pulses simulating human heart pulses to be transmitted to the cells on the culture carrier 11.

Referring to FIG. 2, in certain embodiments, in addition to the two first electrodes 122a, the pulse generating device 12 may further include two second electrodes 122b which connect the piezoelectric element 121 with the actuator 123, and which are disposed opposite to each other in a second direction (Y) transverse to the first direction (X). In this case, when the electrical energy in the form of the PWM signal is applied to the piezoelectric element 121 through the two first electrodes 122a and the two second electrodes 122b, such electrical energy will result in the repetitive contraction and expansion of the piezoelectric element 121 along both the first direction (X) and the second direction (Y).

Referring to FIG. 2 again, in certain embodiments, in addition to the two first electrodes 122a and the two second electrodes 122b, the pulse generating device 12 may further include two third electrodes 122c which connect the piezoelectric element 121 with the actuator 123, and which are disposed opposite to each other in a third direction (Z) transverse to the first direction (X) and the second direction (Y). In this case, when the electrical energy in the form of the PWM signal is applied to the piezoelectric element 121 through the two first electrodes 122a, the two second electrodes 122b and the two third electrodes 122c, such electrical energy will result in the repetitive contraction and expansion of the piezoelectric element 121 along the first direction (X), the second direction (Y), and the third direction (Z).

In some other embodiments, the pulse generating device 12 may be selected from commercially available unimorphs or bimorph piezoelectric devices.

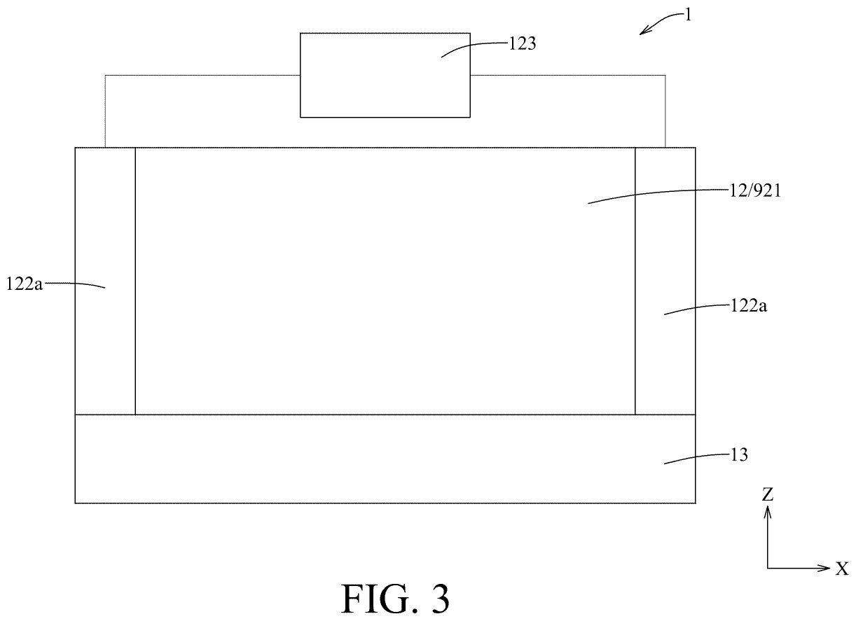

Referring to FIG. 3, in certain embodiments, the culture carrier 11 and the piezoelectric element 121 are integrally formed on a holder 13, and are both made of the piezoelectric material as mentioned in the foregoing. In this case, the pulse generating device 12 may include the two first electrodes 122a only, or may include the two first electrodes 122a and the two second electrodes 122b without the two third electrodes 122c.

Examples of the photonic quasicrystal pattern with multifold symmetry may include, but are not limited to, square-triangular tiling pattern, penrose-tiling pattern, and sunflower pattern.

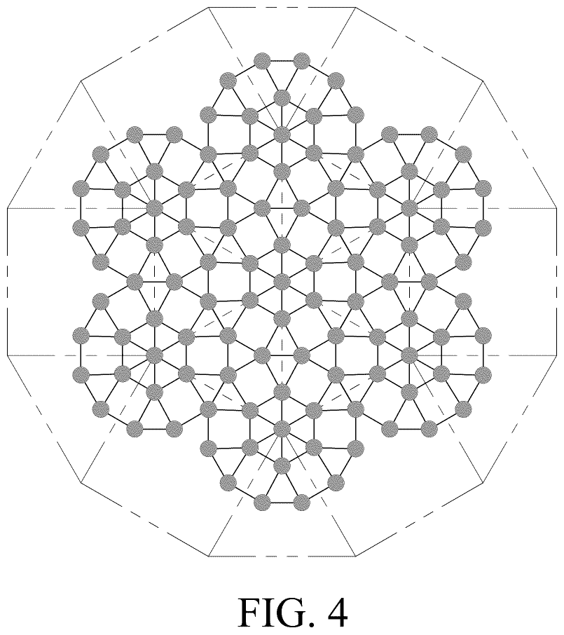

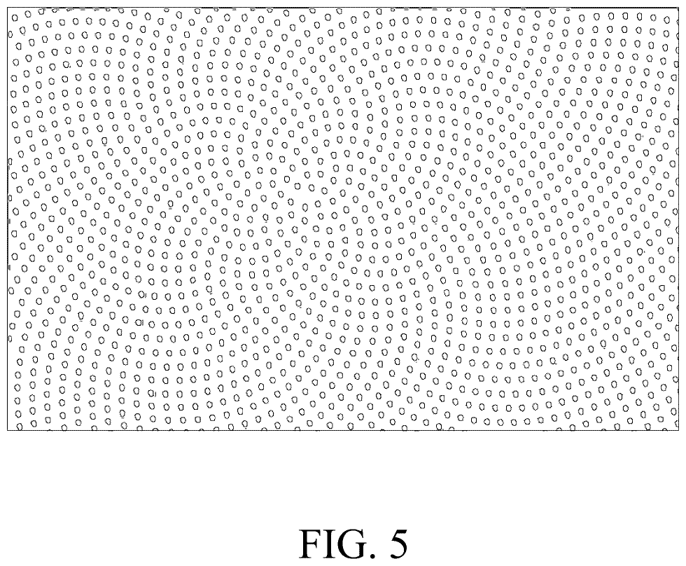

Referring to FIG. 4, in certain embodiments, the photonic quasicrystal pattern is a square-triangular tiling pattern having a 12-fold symmetry or an 8-fold symmetry. Referring to FIG. 5, in certain embodiments, the photonic quasicrystal pattern is a sunflower pattern.

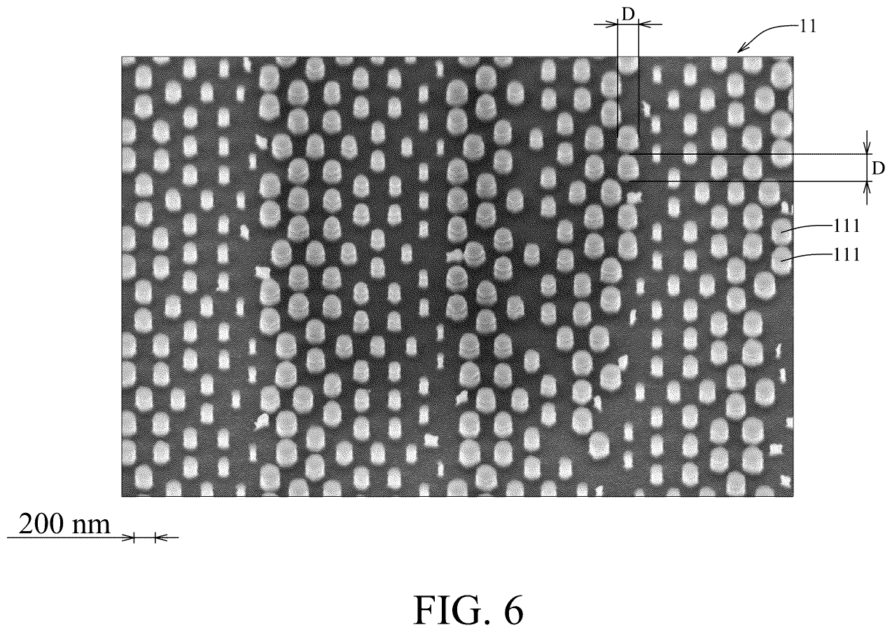

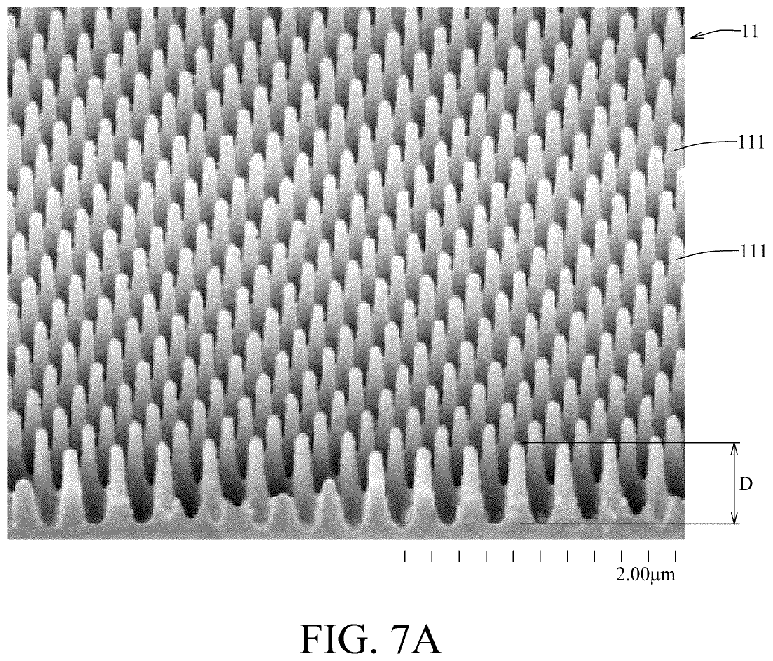

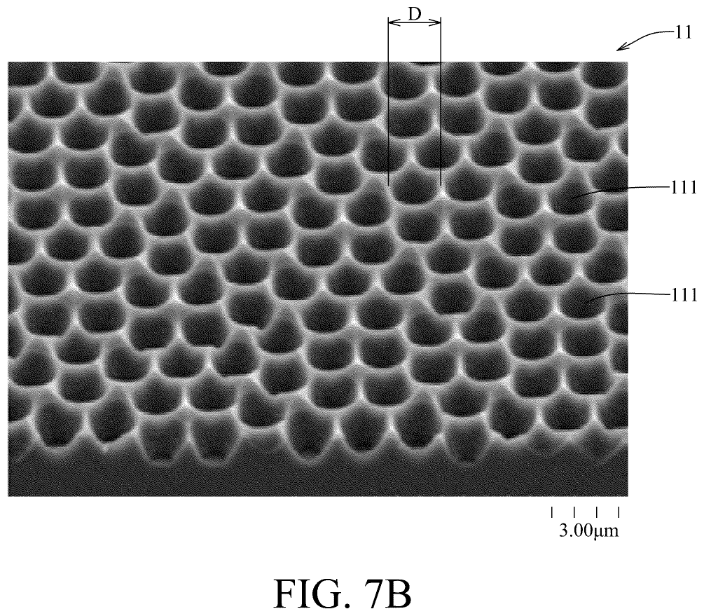

Referring to FIGS. 6, 7A and 7B, in certain embodiments, the culture carrier 11 includes unit elements 111 distributed as the photonic quasicrystal pattern. In certain embodiment, the unit elements 111 are in a form of rods (see FIG. 6). In certain embodiments, the unit elements 111 are in a form of pillars (see FIG. 7A). In other embodiments, the unit elements 11 are in a form of holes (see FIG. 7B). Examples of the shapes of the unit elements 111 may include, but are not limited to, circular, triangular, square, and polyhedron. In some embodiments, the forms of the unit elements 111, i.e., rods, pillars, or holes, may be manipulated to alter the hydrophilicity or hydrophobicity of the surface of the culture carrier 11. For example, certain regions on the surface of the culture carrier 11 may be patterned to be hydrophilic whereas other regions on the surface thereof may be patterned to be hydrophobic.

As shown in FIGS. 6, 7A and 7B, each of the unit elements 111 has a dimension (D) ranging from 50 nm to 2000 nm. The dimension (D) may include height, width or length of each of the unit elements 111.

In certain embodiments, as shown in FIG. 6, the culture carrier 11 has multiple regions, and the unit elements 111 on two adjacent ones of the multiple regions have different dimensions (D) or different orientations.

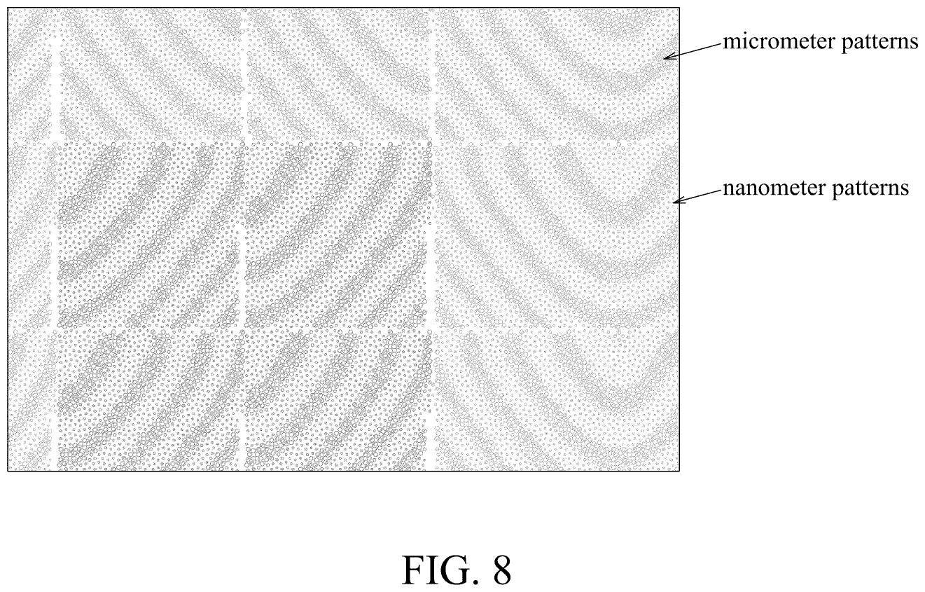

Referring to FIG. 8, in certain embodiments, the culture carrier 11 includes an integrated pattern that includes nanometer-scale patterns and micrometer-scale patterns, both of which are distributed as the photonic quasicrystal pattern. In certain embodiments, the nanometer-scale patterns and micrometer-scale patterns are in a form of rods. In certain embodiments, the nanometer-scale patterns and micrometer-scale patterns are in a form of pillars. In other embodiments, the nanometer-scale patterns and micrometer-scale patterns are in a form of holes. Examples of the shapes of the nanometer-scale patterns and micrometer-scale patterns include, but are not limited to, circular, triangle, square, polyhedrons, and random shapes.

Furthermore, the photonic quasicrystal pattern may be selected from the aforesaid patterns based on the cellular arrangements of living organisms. It should be noted the photonic quasicrystal pattern may be tailored as biomimetic pattern to meet the specific environment for cell growth. For example, cells, e.g., stem cells, may grow and differentiate into specialized cells and/tissues, by selection of a specific type of the photonic quasicrystal pattern in which the unit elements 111 are arranged to imitate the cellular arrangement of cells in living organisms in vivo, such that the stem cells, under the influence of the pulses simulating human heart pulses and in the presence of appropriate nutrients and growth factors, can be selectively grown and/or differentiate into the specified tissue.

It is noted that, since the culture carrier 11 of the apparatus 1 is formed with the photonic quasicrystal pattern imitating cellular arrangement in vivo and electrical pulses with rate variability to simulate real human heart pulses is transmitted to the culture carrier 11 (in other words, the apparatus 1, when in operation, provides a dynamic environment resembling the physiological environment of the human body), the apparatus 1 is deemed useful to enhance cellular growth and/or differentiation in vitro.

Moreover, the present disclosure also provides a method for culturing cells in vitro using the apparatus 1. The method includes steps a) and b). In step a), the cells are placed on the culture carrier 11 which includes the photonic quasicrystal pattern with multifold symmetry. In step b), pulses simulating human heart pulses are transmitted to the cells on the culture carrier 11 while the cells are cultured on the culture carrier 11.

To be specific, in step a), the cells placed on the culture carrier 11 to be cultured thereon are distributed among the unit elements 111, i.e., when the unit elements 111 are in the form of rods or pillars (see FIGS. 6 and 7A), the cells may be received in spaces among the rods or pillars so as to be distributed thereamong, and when the unit elements 111 are in the form of holes (see FIG. 7B), the cells may be received in the holes so as to be distributed thereamong.

According to the present disclosure, the piezoelectric element 121 is disposed under the cells on the culture carrier 11, and in step b), the pulses simulating human heart pulses are generated by providing the PWM signal to the piezoelectric element 121.

In certain embodiments, in step b), the pulses simulating human heart pulses have different frequencies in different time periods, and are respectively generated by providing different PWM signals to the piezoelectric element 121.

According to the present disclosure, the cells are stem cells or organ cells. As used herein, the term “stem cells” refers to undifferentiated biological cells which can differentiate into specialized cells and/or have the ability to divide through mitosis to produce more stem cells. Examples of the stem cells may include, but are not limited to, embryonic stem cells, induced pluripotent stem cells, mesenchymal stem cells, neural stem cells, epithelial stem cells, hepatic stem cells, germ stem cells, hematopoietic stem cells, and skeletal muscle stem cells. The organ cells may be derived from tissue-forming cells. Examples of the organ cells may include, but are not limited to, hepatocytes, renal cells, pulmonary epithelial cells, enterocytes, cardiomyocytes, vascular endothelial cells, skeletal muscle cells, smooth muscle cells, neurons, keratinocytes, adrenal cells, thyroid cells, thymic cells, connective tissue cells, testicular cells, and ovarian cells.

In certain embodiments, optionally, in step b), the cells are cultured in the presence of a heating source or a light source at an optimized temperature. Examples of the light source may include, but are not limited to, ultraviolet A light, ultraviolet B light, visible light, and infrared light. In some other embodiments, the apparatus 11, with the cells placed on the culture carrier 11, is disposed in an incubator with a temperature set at 37° C., such that the cells are cultured at the physiological temperature.

In summary, by virtue of the method for culturing cells in vitro of the present disclosure which utilizes the apparatus 1 that includes the culture carrier 11 formed with the photonic quasicrystal pattern and that is provided with pulses simulating human heart pulses, an environment mimicking the dynamic physiological conditions in the human body is provided for culturing cells, and hence enhancement of cellular growth and/or differentiation in vitro can be achieved.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. An apparatus for culturing cells in vitro, comprising:
a culture carrier for culturing the cells, said culture carrier including a photonic quasicrystal pattern with multifold symmetry; and
a pulse generating device which is capable of generating pulses simulating human heart pulses, said pulse generating device being disposed to transmit the pulses simulating human heart pulses to the cells on said culture carrier.

2. The apparatus as claimed in claim 1, wherein said pulse generating device includes
a piezoelectric element,
two first electrodes connected to said piezoelectric element and disposed opposite to each other in a first direction, and
an actuator connected to said piezoelectric element through said two first electrodes, and being capable of generating an adjustable pulse-width modulation (PWM) signal so as to permit said piezoelectric element to generate the pulses simulating human heart pulses.

3. The apparatus as claimed in claim 2, wherein
said culture carrier and said piezoelectric element are integrally formed and are made of a piezoelectric material that is selected from the group consisting of lead zirconate titanate (PbZrTiO₃), zinc oxide (ZnO), gallium nitride (GaN), polyvinylidene fluoride (PVDF), barium titanate (BaTiO₃), sodium potassium niobate (KNaNbO₃), quartz, ceramic composites, berlinite (AlPO₄), lead titanate (PbTiO₃), lithium niobate (LiNbO₃), lithium tantalite (LiTaO₃), sodium tungstate (Na₂WO₃), bismuth ferrite (BiFeO₃), bismuth titanate (Bi₄Ti₃O₁₂), boron nitride (BN), and combinations thereof; and
said culture carrier is coated with a material selected from biopolymers, proteins, polypeptides, or combinations thereof.

4. The apparatus as claimed in claim 2, wherein said piezoelectric element is made of a piezoelectric material and is disposed beneath said culture carrier, the piezoelectric material being selected from the group consisting of lead zirconate titanate (PbZrTiO₃), zinc oxide (ZnO), gallium nitride (GaN), polyvinylidene fluoride (PVDF), barium titanate (BaTiO₃), sodium potassium niobate (KNaNbO₃), quartz, ceramic composites, berlinite (AlPO₄), lead titanate (PbTiO₃), lithium niobate (LiNbO₃), lithium tantalite (LiTaO₃), sodium tungstate (Na₂WO₃), bismuth ferrite (BiFeO₃), bismuth titanate (Bi₄Ti₃O₁₂), boron nitride (BN), and combinations thereof.

5. The apparatus as claimed in claim 2, wherein said pulse generating device further includes two second electrodes which connect said piezoelectric element with said actuator, and which are disposed opposite to each other in a second direction transverse to the first direction.

6. The apparatus as claimed in claim 5, wherein said pulse generating device further includes two third electrodes which connect said piezoelectric element with said actuator, and which are disposed opposite to each other in a third direction transverse to the first direction and the second direction.

7. The apparatus as claimed in claim 1, wherein said photonic quasicrystal pattern is a square-triangular tiling pattern having a 12-fold symmetry or an 8-fold symmetry.

8. The apparatus as claimed in claim 1, wherein said photonic quasicrystal pattern is a sunflower pattern.

9. The apparatus as claimed in claim 1, wherein said culture carrier includes unit elements distributed as said photonic quasicrystal pattern.

10. The apparatus as claimed in claim 9, wherein said unit elements are in a form of rods, pillars, or holes.

11. The apparatus as claimed in claim 10, wherein each of said unit elements has a shape selected from the group consisting of circular, triangle, square, polyhedrons, and combinations thereof.

12. The apparatus as claimed in claim 9, wherein each of said unit elements has a dimension ranging from 50 nm to 2000 nm.

13. The apparatus as claimed in claim 9, wherein said culture carrier has multiple regions, and said unit elements on two adjacent ones of said multiple regions have different dimensions or different orientations.

14. The apparatus as claimed in claim 1, wherein said culture carrier includes an integrated pattern that includes nanometer-scale patterns and micrometer-scale patterns, said nanometer-scale patterns and micrometer-scale patterns being distributed as said photonic quasicrystal pattern.

15. The apparatus as claimed in claim 14, wherein said nanometer-scale patterns and micrometer-scale patterns are in a form of rods, pillars or holes each having a shape selected from the group consisting of circular, triangle, square, polyhedrons, random shapes, or combinations thereof.

16. A method for culturing cells in vitro, comprising the steps of:
a) placing the cells on a culture carrier, the culture carrier including a photonic quasicrystal pattern with multifold symmetry; and
b) transmitting pulses simulating human heart pulses to the cells on the culture carrier while culturing the cells on the culture carrier.

17. The method as claimed in claim 16, wherein in step b), the pulses simulating human heart pulses are generated by providing an adjustable pulse-width modulation (PWM) signal to a piezoelectric element, the piezoelectric element being disposed under the cells on the culture carrier.

18. The method as claimed in claim 16, wherein in step b), the pulses simulating human heart pulses have different frequencies in different time periods, and are respectively generated by providing different pulse-width modulation (PWM) signals to a piezoelectric element, the piezoelectric element being disposed under the cells on the culture carrier.

19. The method as claimed in claim 16, wherein the cells are stem cells or organ cells.

20. The method as claimed in claim 16, wherein in step b), the cells are cultured in the presence of a heating source or a light source, the light source being selected from the group consisting of ultraviolet A light, ultraviolet B light, visible light, infrared light, and combinations thereof.
